(19) United States
(12) Patent Application Publication
(10) Pub. No.: US 2024/0274302 A1
(43) Pub. Date: Aug. 15, 2024

(54) System of Predicting Sensitivity of Klebsiella Against MeropeneM and Method

(71) Applicants: Peking Union Medical College Hospital, Chinese Academy of Medical Sciences, Beijing (CN); Hangzhou Jieyi Biotechnology Company Limited, Hanzhou City (CN)

(72) Inventors: Qiwen Yang, Beijing (CN); Jing CHEN, Beijing (CN); Wei Yu, Beijing (CN); Jun Wang, Beijing (CN); Xiaobing Chu, Beijing (CN); Shiyu Chen, Beijing (CN); Qian Zhang, Beijing (CN); Yao Yao, Beijing (CN)

(21) Appl. No.: 18/523,698

(22) Filed: Nov. 29, 2023

(30) Foreign Application Priority Data
Feb. 6, 2023 (CN) 202310065167.5

(51) Int. Cl. G16H 70/60

(52) U.S. Cl. CPC G16H 70/60

(57) ABSTRACT

Disclosed are a system and method of predicting sensitivity of Klebsiella against MeropeneM, which belong to bioinformatics art. The system comprises a computer readable storage medium on which is stored a computer program. An Exp (−k) power value calculation method is implemented when said computer program is executed by a processor. Said Exp(−k) power value calculation method comprises following computing steps: S1: k value is calculated according to formula I:

$$Formula I$$

$$\text{?} - 1.44 + 0. \text{?} \times ( \frac{\text{?}}{\text{?}} ) - 0.66 \times ( \frac{\text{?}}{\text{?}} ) + 0. \text{?} \times ( \frac{\text{?}}{\text{?}} ) + 3.04 \text{?} \times ( \frac{\text{?}}{\text{?}} ) - 0.46 \times ( \frac{\text{?}}{\text{?}} )$$

$$\text{?} \text{indicates text missing or illegible when filed}$$

S2: Exp(−k) power value with natural constant e as base and −k as exponent is calculated; wherein, C1-C5 are respectively the number of mphA, marA, Klebsiella pneumoniae KpnE, KPC-1, floR gene copies in the candidate Klebsiella strain. The accuracy of predicting sensitivity of Klebsiella against meropenem using the prediction method and prediction system of the present invention is about 99.4%.

### CROSS-REFERENCE TO RELATED APPLICATION

This application claims the benefit of and priority to Chinese patent application 202310065167.5 filed Feb. 6, 2023, which is incorporated herein by reference in its entirety.

### BACKGROUND

Antibiotics were once a “secret weapon” for humans to fight against many diseases. In the late 19th and early 20th centuries, the discovery of a series of antibiotics greatly increased human lifespan. In recent years, with the continuous application of antibiotics, drug abuse has gradually emerged, which leads to an increase in clinical antibiotic resistance and adverse reactions, and brings a heavy burden to the global economy. Effectively controlling the abuse of antibiotics in healthcare is an important link in addressing the global issue of antibiotic resistance.

Pathogenic microorganisms refer to microorganisms that can invade the human body, and cause infections or even infectious diseases, which are also known as pathogens. Pathogenic microorganisms mainly include bacteria, viruses, fungi, parasites, mycoplasma, chlamydia, rickettsia, spirochetes, etc. There are various types of microbial samples. Intestinal samples include feces, mucous membranes, etc. Liquid samples include urine, blood, cerebrospinal fluid, saliva, sputum, alveolar lavage fluid, amniotic fluid, etc. Swab samples include samples from oral cavity, reproductive tract, skin, etc. Other samples include tissues, liver, eyes, placenta, etc.

The Latin scientific name of Genus Klebsiella is Klebsiella Trevisan, and its systematic classification level is Genus. It is a straight bacterium with a diameter of 0.3-1.0 μm. 0.6-6.0 in length μM, and it's single, paired, or short chain arrangement. Currently reported strains of this genus include Klebsiella pneumoniae, Klebsiella aerogenes, Klebsiella oxytoca, Klebsiella quasipneumoniae, Klebsiella variicola, Klebsiella michiganensis, etc.

Among them, Klebsiella pneumoniae, as a model strain of the genus Genus Klebsiella, is widely present in the environment, and is a common opportunistic pathogen that easily colonizes the respiratory and intestinal tracts of patients, and causes infections in multiple parts of the digestive tract, respiratory tract, blood, etc. It is one of the pathogenic bacteria that cause human pneumonia and is also one of the common drug-resistant bacteria in hospitals. According to a study by the Second Military Medical University, the resistance rate against meropenem of carbapenem resistant Klebsiella pneumoniae isolated from 2014 to 2017 to was 62.5% (252/403).

Meropenem is a widely antibacterial and injectable antibiotic, which is used to treat various infections including meningitis and pneumonia. It is a type of β Lactam antibiotics and belongs to the category of carbapenems. Meropenem has a wide antibacterial spectrum, strong antibacterial activity, and is stable to βlactamase.

Bacterial drug sensitivity testing is currently the most commonly used method for detecting bacterial resistance in clinic and laboratory both domestically and internationally. There are methods such as paper disc method, agar dilution method, broth dilution method, and concentration gradient method. Except for the paper disc method, all other methods can obtain relatively accurate minimum inhibitory concentration (MIC) of drugs. It's required firstly to obtain pure culture in the bacterial drug sensitivity test, which is not suitable for difficult to cultivate and non cultured bacteria, and takes a long time. Sometimes, it is difficult to meet the current clinical needs for rapid diagnosis and targeted treatment of severe and emergency infections. The traditional detection and identification methods of pathogenic microorganisms fail to meet the comprehensive needs of wide coverage, speed, and accuracy. The diagnosis and treatment of infectious diseases are mainly based on empirical and directional methods. Clinical doctors and patients urgently need innovative detection methods to identify infectious pathogens more comprehensively, accurately, and quickly, which can assist in diagnosis and reasonable standardized medication treatment, shorten treatment courses, reduce mortality rates, and reduce medical costs.

With the promotion of emerging technologies such as PCR technology, whole genome sequencing technology, microfluidic technology, VITEK-2 compact fully automated bacterial identification/drug sensitivity system, the exploration of new technologies for bacterial resistance detection is gradually deepening, and various new technologies and methods for bacterial resistance detection are becoming increasingly mature. Although the VITEK-2 compact fully automated bacterial identification/drug sensitivity system is simple and fast, its accuracy in bacterial identification/drug sensitivity evaluation is influenced by the sample status and bacterial culture, and its usage cost is relatively high.

Therefore, there is an urgent need to develop a system and method in this field that can quickly, accurately, and cost-effectively predict the sensitivity of Klebsiella strains against meropenem.

### SUMMARY

In response to the aforementioned shortcomings and requirements of prior art in this field, the present invention aims to provide a system and method of predicting sensitivity of Klebsiella against MeropeneM.

Technical solution of the present invention is as follows.

A system of predicting sensitivity of Klebsiella against MeropeneM, comprises a computing unit. The computing unit comprises a computer readable storage medium on which is stored a computer program. An Exp (−k) power value calculation method is implemented when the computer program is executed by a processor. The Exp(−k) power value calculation method comprises following computing steps:
S1: k value is calculated according to formula I:

$$k = - 1.44 + 0.708 \times ( \frac{\text{?}}{\text{?}} ) - 0.66 \times ( \frac{\text{?}}{\text{?}} ) + 0.088 \times ( \frac{\text{?}}{\text{?}} ) + 3.048 \times ( \frac{\text{?}}{\text{?}} ) - 0.46 \times ( \frac{\text{?}}{\text{?}} ) \\ k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - 0.46 \times ( \frac{C 5 - 0.196}{0.67} ) \qquad Formula I$$

$$\text{?} \text{indicates text missing or illegible when filed}$$

S2: Exp(−k) power value with natural constant e as base and −k as exponent is calculated.

In formula I,
C1 is the number of mphA gene copies in a candidate Klebsiella strain,
C2 is the number of marA gene copies in a candidate Klebsiella strain,
C3 is the number of Klebsiella pneumoniae KpnE gene copies in a candidate Klebsiella strain,
C4 is the number of KPC-1 gene copies in a candidate Klebsiella strain,
C5 is the number of floR gene copies in a candidate Klebsiella strain.

In above formula I, the first pair of parentheses includes C1 minus 0.223 divided by 0.757; the second pair of parentheses includes C2 minus 0.951 divided by 0.090; the third pair of parentheses includes C3 minus 0.952 divided by 0.103; the fourth pair of parentheses includes C4 minus 0.469 divided by 1.090; and the fifth pair of parentheses includes C5 minus 0.196 divided by 0.670.

In above formula I, k=0−1.44+0.708×(C1 minus 0.223 divided by 0.757)−0.660×(C2 minus 0.951 divided by 0.090)+0.088×(C3 minus 0.952 divided by 0.103)+3.048×(C4 minus 0.469 divided by 1.090)−0.460×(C5 minus 0.196 divided by 0.670).

The system of predicting sensitivity of Klebsiella against MeropeneM, also comprises a result output unit. The computing unit transmits the calculated Exp(−k) power value to the result output unit. The result output unit recognizes Exp(−k) power value and outputs result;
preferably, said natural constant e=2.718281828459045.
the result output unit outputs resistant result R when recognizing Exp(−k) power value <1;
the result output unit outputs sensitive result S when recognizing Exp(−k) power value ≥1;
the result output unit and the computing unit are communicated through data-path, Exp(−k) power value calculated by the computing unit is transmitted to the result output unit;
preferably, said sensitive result S refers to that the candidate Klebsiella strain is sensitive to MeropeneM, and said resistant result R refers to that the candidate Klebsiella strain is resistant against MeropeneM.

The system of predicting sensitivity of Klebsiella against MeropeneM, also comprises an experiment unit and a data input unit. The experiment unit and data input unit are communicated through a data-path. The experiment unit outputs experiment results which are transmitted to the data input unit and transformed to independent variables, The data input unit and computing unit are communicated through the data-path, Independent variables are transmitted to the computing unit.

The independent variables include: values of C1, C2, C3, C4, C5;
preferably, said experiment results comprise: the number of mphA gene copies in the candidate Klebsiella strain, the number of marA gene copies in the candidate Klebsiella strain, the number of Klebsiella pneumoniae KpnE gene copies in the candidate Klebsiella strain, the number of KPC-1 gene copies in the candidate Klebsiella strain, the number of floR gene copies in the candidate Klebsiella strain.

A method of predicting sensitivity of Klebsiella against MeropeneM, comprises:
S1: k value is calculated according to formula I:

$$k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - 0.46 \times ( \frac{C 5 - 0.196}{0.67} ) \qquad Formula I$$

S2: Exp(−k) power value with natural constant e as base and −k as exponent is calculated.

In formula I,
C1 is the number of mphA gene copies in a candidate Klebsiella strain,
C2 is the number of marA gene copies in a candidate Klebsiella strain,
C3 is the number of Klebsiella pneumoniae KpnE gene copies in a candidate Klebsiella strain,
C4 is the number of KPC-1 gene copies in a candidate Klebsiella strain,
C5 is the number of floR gene copies in a candidate Klebsiella strain.

In above formula I, the first pair of parentheses includes C1 minus 0.223 divided by 0.757; the second pair of parentheses includes C2 minus 0.951 divided by 0.090; the third pair of parentheses includes C3 minus 0.952 divided by 0.103; the fourth pair of parentheses includes C4 minus 0.469 divided by 1.090; and the fifth pair of parentheses includes C5 minus 0.196 divided by 0.670.

In above formula I, k=0−1.44+0.708×(C1 minus 0.223 divided by 0.757)−0.660×(C2 minus 0.951 divided by 0.090)+0.088×(C3 minus 0.952 divided by 0.103)+3.048×(C4 minus 0.469 divided by 1.090)−0.460×(C5 minus 0.196 divided by 0.670).

A predicting result corresponding to Exp(−k) power value<1 is the candidate Klebsiella strain sensitive to MeropeneM, and a predicting result corresponding to Exp(−k) power value≥1 is the candidate Klebsiella strain resistant against MeropeneM.
said natural constant e=2.718281828459045;
the number of mphA, marA, Klebsiella pneumoniae KpnE, KPC-1, floR gene copies in the candidate Klebsiella strain are obtained through a second-generation high-throughput sequencing method;
the number of gene copies in the candidate Klebsiella strain-depth of gene contigs/depth of genome contigs;
preferably, said genome contigs is a longest contigs segment assembled from sequencing results by SPAdes v3.13.0 software;
said depth of genome contigs is a depth of genome contigs calculated by SPAdes v3.13.0 software;
said depth of gene contigs refers to a sum of depths of each contig which has said gene copies and said gene is located on;
preferably, each contig which has said gene copies is annotated by blat (v. 36) software and diamond (v2.0.4.142) software through CARD database alignment between said gene cds and protein sequence;
preferably, depths of each contig which has said gene copies and said gene is located on are calculated through SPAdes v3.13.0 software.

One aspect of the present invention proposes a method of predicting sensitivity of Klebsiella against MeropeneM.

In this invention, after routine processing of the obtained microbial samples, necessary steps such as DNA extraction and sequencing can be carried out. Through bioinformatics process analysis, the state of the relevant features of the Klebsiella prediction system in the samples can be obtained. The feature state information can be imported into the system to predict the drug sensitivity of the samples. Compared to traditional methods, it has advantages such as simple operation, short detection time, and accurate species identification.

In order to effectively evaluate the performance of a prediction system, it is necessary to establish a dataset that is not involved in the establishment of the prediction system, and evaluate the accuracy of the prediction system on this dataset. This independent dataset is called the test set. The evaluation methods for system prediction effectiveness include F1 score, Precision, Recall, and confusion matrix.

The method of the present invention also has the following advantages. The present invention utilizes a test set to evaluate the accuracy of the system. The average accuracy of the method is 0.994, F1 score is 0.984, and recall score is 0.968. On the one hand, the present invention is less affected by subjective factors from such as operators, and has good detection stability; on the other hand, it achieves rapid and accurate identification of infectious pathogens and prediction of drug sensitivity of the test samples, meanwhile, it assists in diagnosis and rational & standardized medication treatment, has high throughput, and reduce medical costs.

### DESCRIPTION OF DRAWINGS

FIG. 1 is a schematic diagram of the structure of the drug resistance prediction system provided by some examples of the present invention (in dashed boxes) and its workflow diagram.

FIG. 2 is a schematic diagram of the structure of the drug resistance prediction system provided by other examples of the present invention (in dashed boxes) and its workflow diagram.

### DETAILED DESCRIPTION

In order to facilitate the understanding of the present invention, a more comprehensive description of the present invention will be provided in the embodiments below.

Unless otherwise defined, all technical and scientific terms used in this article have the same meanings as those commonly understood by a person skilled in the art of the present invention. The terms used in the specification of the present invention in this article are only for the purpose of describing specific embodiments and are not intended to limit the present invention.

The reagents used in the following embodiments are commercially available unless otherwise specified.

### Sources of Biomaterials

The 160 samples used in the experimental example of the present invention are pure cultures of Klebsiella strains isolated from clinical blood culture, from Beijing Union Medical College Hospital of the Chinese Academy of Medical Sciences.

All tested strains (strains) were identified as Klebsiella (scientific name: Genus Klebsiella, Latin name: Klebsiella Trevisan, systematic classification level: Genus) by mass spectrometry MALDI-TOF MS.

On the Illumina Novaseq NGS sequencing platform, these strains include 122 Klebsiella pneumoniae strains, 18 Klebsiella aerogenes strains, 7 Klebsiella oxytoca strains, 6 Klebsiella quasipneumoniae strains, 4 Klebsiella variicola strains, and 3 Klebsiella michiganensis strains, all of them are reported strains of the Klebsiella genus.

The above strains or strains can be obtained from common cases of Klebsiella pneumoniae pneumonia or from the applicant's laboratory. The applicant promises to distribute strains to the public within 20 years from the application date of the present invention for verifying the technical effects of the present invention.

### Examples Group 1. System of Resistance Predicting System

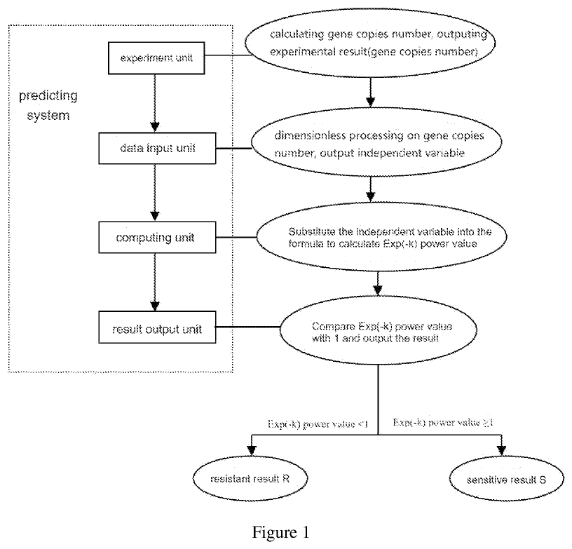

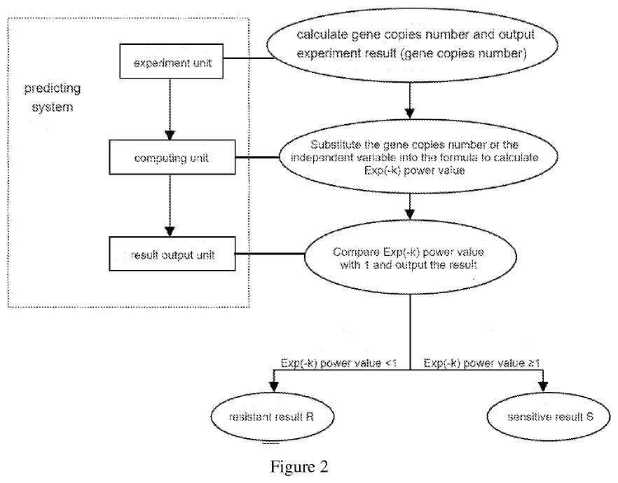

This group of examples provides a system of predicting sensitivity of Klebsiella against MeropeneM. All examples of this group possesses the following common features: as shown in FIG. 1 and FIG. 2, the system of predicting sensitivity of Klebsiella against MeropeneM comprising: computing unit; said computing unit comprises a computer readable storage medium on which is stored a computer program, characterized in that, an Exp (−k) power value calculation method is implemented when said computer program is executed by a processor; said Exp(−k) power value calculation method comprises following computing steps:
S1: k value is calculated according to formula I:

$$k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - \\ 0.46 \times ( \frac{C 5 - 0.196}{0.67} ) \qquad Formula I$$

S2: Exp(−k) power value with natural constant e as base and −k as exponent is calculated.

In formula I,
C1 is the number of mphA gene copies in a candidate Klebsiella strain,
C2 is the number of marA gene copies in a candidate Klebsiella strain,
C3 is the number of Klebsiella pneumoniae KpnE gene copies in a candidate Klebsiella strain,
C4 is the number of KPC-1 gene copies in a candidate Klebsiella strain,
C5 is the number of floR gene copies in a candidate Klebsiella strain.

In above formula I, the first pair of parentheses includes C1 minus 0.223 divided by 0.757; the second pair of parentheses includes C2 minus 0.951 divided by 0.090; the third pair of parentheses includes C3 minus 0.952 divided by 0.103; the fourth pair of parentheses includes C4 minus 0.469 divided by 1.090; and the fifth pair of parentheses includes C5 minus 0.196 divided by 0.670.

In above formula I, k=0−1.44+0.708×(C1 minus 0.223 divided by 0.757)−0.660×(C2 minus 0.951 divided by 0.090)+0.088×(C3 minus 0.952 divided by 0.103)+3.048×(C4 minus 0.469 divided by 1.090)−0.460×(C5 minus 0.196 divided by 0.670).

In some examples of this invention, said natural constant e=2.718281828459045.

In more specific examples, above genes are all known genes reported in the art, specifically as follows:
mphA gene is mphA gene recorded in “Molecular Characterization of a Multidrug-Resistant Klebsiella pneumoniae Strain R46 Isolated from a Rabbit”.
marA gene is marA gene recorded in “Correlation of the expression of acrB and the regulatory genes marA, soxS and ramA with antimicrobial resistance in clinical isolates of Klebsiella pneumoniae endemic to New York City”.

Klebsiella pneumoniae KpnE gene is Klebsiella pneumoniae KpnE gene recorded in “An unequivocal superbug: PDR Klebsiella pneumoniae with an arsenal of resistance and virulence factor genes”.

KPC-1 gene is KPC-1 gene recorded in “Novel Carbapenem-Hydrolyzing b-Lactamase, KPC-1, from a Carbapenem-Resistant Strain of Klebsiella pneumoniae”.

floR gene is floR gene recorded in “Spread of the florfenicol resistance floR gene among clinical Klebsiella pneumoniae isolates in China”.

In further examples, as shown in FIG. 1 and FIG. 2, the system of predicting sensitivity of Klebsiella against MeropeneM, also comprising: result output unit; said result output unit outputs sensitive result or resistant result; said sensitive result S refers to that the candidate Klebsiella strain is sensitive to MeropeneM, and said resistant result R refers to that the candidate Klebsiella strain is resistant against MeropeneM.
the result output unit outputs resistant result R when Exp(−k) power value <1;
the result output unit outputs sensitive result S when Exp(−k) power value ≥1; Preferably, the result output unit and the computing unit are communicated through data-path;

Preferably, Exp(−k) power value calculated by the computing unit is transmitted to the result output unit.

In more further example, as shown in FIG. 1, the system of predicting sensitivity of Klebsiella against MeropeneM, also comprising: experiment unit and data input unit;
the experiment unit and data input unit are communicated through data-path; the experiment unit outputs experiment results which are transmitted to the data input unit and transformed to independent variables;
said data input unit and computing unit are communicated through data-path;
independent variables are transmitted to the computing unit through data-path.

In more specific example, said data-path is data transmission carrier well-known to a person skilled in the arts of computer and electronics. The data path can be selected from wired or wireless form, for example, it can be a wired path, line, wireless path, WiFi connection, wireless channel, etc.

Preferably, said independent variables include: values of C1, C2, C3, C4, C5;

Preferably, said experiment results comprise: the number of mphA, marA, Klebsiella pneumoniae KpnE, KPC-1, floR gene copies in the candidate Klebsiella strain.

The copy number of known genes in known strains can be routinely obtained by a person skilled in the arts of molecular biology and bioinformatics through conventional techniques such as sequencing and bioinformatics analysis. The mphA, marA, Klebsiella pneumoniae KpnE, KPC-1, and floR genes involved in the experiment results output by the experiment unit of the prediction system of the present invention are all reported genes in the art, and their gene information and primary structural sequences can be queried through the NCBI website or other known bioinformatics databases. By conducting whole genome sequencing of the candidate Klebsiella strain, the numbers of each of the aforementioned genes copies in the strain can be obtained.

In other specific examples, the number of mphA, marA, Klebsiella pneumoniae KpnE, KPC-1, floR gene copies in the candidate Klebsiella strain are obtained through a second-generation high-throughput sequencing method.

In more specific examples, the number of gene copies in the candidate Klebsiella strain=depth of gene contigs/depth of genome contigs;
preferably, said genome contigs is a longest contigs segment assembled from sequencing results by SPAdes v3.13.0 software;
said depth of genome contigs is a depth of genome contigs calculated by SPAdes v3.13.0 software;
said depth of gene contigs refers to a sum of depth of each contig which has said gene copies and said gene is located on;
preferably, each contig which has said gene copies is annotated by blat (v. 36) software and diamond (v2.0.4.142) software through CARD database alignment between said gene cds and protein sequence;
preferably, depth of each contig which has said gene copies and said gene is located on are calculated through SPAdes v3.13.0 software.

The second-generation high-throughput sequencing method has a conventional technical meaning that is well-known to a person skilled in the art, while obtaining gene copy numbers using the second-generation high-throughput sequencing method is a conventional technical means that is well-known to a person skilled in the art. In some specific embodiments, the specific method for calculating the gene copy number is as follows:

Sequencing of strains is conducted by using second-generation high-throughput sequencing methods. The average sequencing depth is about 150×, and the approximate sequencing amount for the Klebsiella genus is about 1G. Using the depth of contigs obtained and calculated by SPAdes (v3.13.0) assembly software during the assembly process as the standard, the longest contigs segment is defined as the genome segment. Prokka software (1.14.6) is used to conduct gene predicting on contigs, and all gene cds and protein sequences on contigs are obtained. blat (v. 36) software and Diamond (v2.0.4.142) software are respectively used to compare the cds and protein sequences in the CARD database, sequences with a similarity greater than 90% are positive sequences, and annotation results for all resistance genes are obtained. The number of all gene copies on contigs are calculated using formula II as follows:

Formula II: the number of all gene copies on said contigs=depth of said contigs/depth of genome contigs

If a gene has two or more genome copies on different or the same contigs, the final number of gene copies is equal to the sum of all calculated number of the gene copies. Examples of calculation methods are as follows:
assuming that there is only one copy of the KPC-1 gene on all contigs, the number of the KPC-1 gene copies is:
the number of KPC-1 gene copies=depth of contigs which KPC-1 gene is located on/depth of genome contigs.

Assuming that the KPC-1 gene has 2 copies on one contigs and no copies on other contigs, the number of the KPC-1 gene copies is:
the number of KPC-1 gene copies-2X depth of contigs which KPC-1 gene is located on/depth of genome contigs.

Assuming that the KPC-1 gene has one copy on one contig1 and contig2, but no copies on other contigs, the number of the KPC-1 gene copies is:
the number of KPC-1 gene copies=depth of contig 1 which KPC-1 gene is located on/depth of genome contigs+ depth of contig 2 which KPC-1 gene is located on/depth of genome contigs.

In more specific embodiments, the result output unit, experiment unit, and data input unit are all set as computer readable storage media on which computer programs are stored.

In some embodiments, when the computer program on the computer readable storage medium of the result output unit is executed by the processor, a method of comparing the Exp (−k) power value with 1 is implemented and the result is output;

That method of comparing the Exp (−k) power value with 1 is implemented and the result is output refers to:
the result output unit outputs resistant result R when Exp(−k) power value <1;
the result output unit outputs sensitive result S when Exp(−k) power value ≥1;

In other embodiments, a method for calculating the number of gene copies is implemented when the computer program on the computer readable storage medium of the experiment unit is executed by the processor;

The method for calculating the number of gene copies is a conventional technical means well known to a person skilled in the art, and the specific steps are as follows:
S1: Take the maximum from depth of the genome contigs calculated by SPAdes v3.13.0 assembly software to obtain the genome contigs;
S2: Use BLAT (v. 36) software and Diamond (v2.0.4.142) software to compare the CDs and protein sequences of a certain gene in the CARD database, and obtain each contigs with that gene copies by annotation;
S3: SPAdes v3.13.0 assembly software calculates the depth of the gene on each contigs has the gene copies;
S4: Calculate the sum of the depths of the gene on each contigs has the gene copies to obtain the depth of the contigs where the gene is located;
S5: Calculate the number of the gene copies according to the following formula, the number of copies=depth of gene contigs/depth of genome contigs.

In some embodiments, the computer program on the computer readable storage medium of the data input unit is executed by the processor to achieve dimensionless processing of the number of gene copies.

The dimensionless processing involves removing data dimensions or data units from the number of gene copies to obtain dimensionless values. Generally speaking, the data dimension or unit of the number of gene copies is: copy, number, or copies. In other embodiments, as shown in FIG. 2, the system of predicting sensitivity of Klebsiella against meropenem may not require a data input unit. The experiment unit is directly connected to the computing unit through a data-path, allowing the number of gene copies (experiment results) or independent variable data calculated by the experiment unit to be directly input into the computing unit for Exp (−k) power value calculation.

### Examples Group 2. The Method of Predicting Klebsiella Resistant Against MeropeneM of this Invention

This group examples provides a method of predicting sensitivity of Klebsiella against meropenem. All examples of this group possesses the following common features:
S1: k value is calculated according to formula I:

$$k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - \\ 0.46 \times ( \frac{C 5 - 0.196}{0.67} ) \qquad Formula I$$

$$k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - 0.46 \times ( \frac{C 5 - 0.196}{0.67} )$$

S2: Exp(−k) power value with natural constant e as base and −k as exponent is calculated.

In formula I,
C1 is the number of mphA gene copies in a candidate Klebsiella strain,
C2 is the number of marA gene copies in a candidate Klebsiella strain,
C3 is the number of Klebsiella pneumoniae KpnE gene copies in a candidate Klebsiella strain,
C4 is the number of KPC-1 gene copies in a candidate Klebsiella strain,
C5 is the number of floR gene copies in a candidate Klebsiella strain.

In above formula I, the first pair of parentheses includes C1 minus 0.223 divided by 0.757; the second pair of parentheses includes C2 minus 0.951 divided by 0.090; the third pair of parentheses includes C3 minus 0.952 divided by 0.103; the fourth pair of parentheses includes C4 minus 0.469 divided by 1.090; and the fifth pair of parentheses includes C5 minus 0.196 divided by 0.670.

In above formula I, k=0−1.44+0.708×(C1 minus 0.223 divided by 0.757)−0.660×(C2 minus 0.951 divided by 0.090)+0.088×(C3 minus 0.952 divided by 0.103)+3.048×(C4 minus 0.469 divided by 1.090)−0.460×(C5 minus 0.196 divided by 0.670).

A predicting result corresponding to Exp(−k) power value<1 is the candidate Klebsiella strain sensitive to MeropeneM, and a predicting result corresponding to Exp(−k) power value≥1 is the candidate Klebsiella strain resistant against MeropeneM.

In above formula I, e, as a mathematical constant, is the base of a natural logarithmic function, also known as a natural constant, natural base, or Euler number. It is an infinite non recurring decimal, which has the conventional technical meaning commonly understood by a ordinary technical person skilled in the art of mathematics. Its value is approximately: e=2.71828182845904523536.

In some examples of this invention, the value of said natural constant e is 2.718281828459045;

In some specific examples, the number of mphA, marA, Klebsiella pneumoniae KpnE, KPC-1, floR gene copies in the candidate Klebsiella strain are obtained through a second-generation high-throughput sequencing method.

In more specific examples, the number of gene copies in the candidate Klebsiella strain-depth of gene contigs/depth of genome contigs;
preferably, said genome contigs is a longest contigs segment assembled from sequencing results by SPAdes v3.13.0 software;
said depth of genome contigs is a depth of genome contigs calculated by SPAdes v3.13.0 software;
said depth of gene contigs refers to a sum of depths of each contig which has said gene copies and said gene is located on;
preferably, each contig which has said gene copies is obtained by annotation with blat (v. 36) software and diamond (v2.0.4.142) software through CARD database alignment between said gene cds and protein sequence;
preferably, depths of each contigs which has said gene copies and said gene is located on are calculated through SPAdes v3.13.0 software.

The second-generation high-throughput sequencing method has a conventional technical meaning that is well-known to a person skilled in the art, while obtaining the number of gene copies by using the second-generation high-throughput sequencing method is a conventional technical means that is well-known to a person skilled in the art.

In some specific embodiments, the specific method for calculating the number of gene copies is as follows:

Sequencing of strains is conducted by using second-generation high-throughput sequencing methods. The average sequencing depth is about 150×, and the approximate sequencing amount for the Klebsiella genus is about 1G. Using the depth of contigs obtained and calculated by SPAdes (v3.13.0) assembly software during the assembly process as the standard, the longest contigs segment is defined as the genome segment. Prokka software (1.14.6) is used to conduct gene predicting on contigs, and all gene cds and protein sequences on contigs are obtained. blat (v. 36) software and Diamond (v2.0.4.142) software are respectively used to compare the cds and protein sequences in the CARD database, sequences with a similarity greater than 90% are positive sequences, and annotation results for all resistance genes are obtained. The number of all gene copies on contigs are calculated using formula II as follows:

Formula II: the number of all gene copies on said contigs=depth of said contigs/depth of genome contigs

If a gene has two or more genome copies on different or the same contigs, the final number of gene copies is equal to the sum of all calculated number of the gene copies. Examples of calculation methods are as follows:
assuming that there is only one copy of the KPC-1 gene on all contigs, the number of the KPC-1 gene copies is:
the number of KPC-1 gene copies=depth of contigs which KPC-1 gene is located on/depth of genome contigs.

Assuming that the KPC-1 gene has 2 copies on one contigs and no copies on other contigs, the number of the KPC-1 gene copies is:
the number of KPC-1 gene copies=2× depth of contigs which KPC-1 gene is located on/depth of genome contigs.

Assuming that the KPC-1 gene has one copy on one contig1 and contig2, but no copies on other contigs, the number of the KPC-1 gene copies is:
the number of KPC-1 gene copies=depth of contig 1 which KPC-1 gene is located on/depth of genome contigs+ depth of contig 2 which KPC-1 gene is located on/depth of genome contigs.

### Experimental Example. Performance Evaluation on Predicting System and Predicting Method of this Invention

The prediction system of the present invention was evaluated using 160 clinical samples, and the comparison between the broth micro dilution classification results and the system prediction results of 160 clinical samples is shown in Table 1. In the table below, S represents sensitivity and R represents resistance.

TABLE 1

| Sample | | result of | Result of Broth micro |
|---|---|---|---|
| NO. | Exp(−k) | predicting system | dilution method |
| s1 | 0.005025126 | R | R |
| s2 | 7.695652174 | S | S |
| s3 | 18.60784314 | S | S |
| s4 | 18.60784314 | S | S |
| s5 | 22.25581395 | S | S |
| s6 | 22.25581395 | S | S |
| s7 | 22.25581395 | S | S |
| s8 | 23.3902439 | S | S |
| s9 | 22.25581395 | S | S |
| s10 | 10.11111111 | S | S |
| s11 | 18.23076923 | S | S |
| s12 | 22.25581395 | S | S |
| s13 | 24.64102564 | S | S |
| s14 | 23.3902439 | S | S |
| s15 | 8.433962264 | S | S |
| s16 | 19.83333333 | S | S |
| s17 | 22.25581395 | S | S |
| s18 | 30.25 | S | S |
| s19 | 22.25581395 | S | S |
| s20 | 22.80952381 | S | S |
| s21 | 8.259259259 | S | S |
| s22 | 22.25581395 | S | S |
| s23 | 0.10864745 | R | R |
| s24 | 16.24137931 | S | S |
| s25 | 0.023541453 | R | R |
| s26 | 0.027749229 | R | R |
| s27 | 0.886792453 | R | R |
| s28 | 22.25581395 | S | S |
| s29 | 22.25581395 | S | S |
| s30 | 18.60784314 | S | S |
| s31 | 22.25581395 | S | S |
| s32 | 19.40816327 | S | S |
| s33 | 0.015228426 | R | R |
| s34 | 16.24137931 | S | S |
| s35 | 14.15151515 | S | S |
| s36 | 22.25581395 | S | S |
| s37 | 0.092896175 | R | R |
| s38 | 22.25581395 | S | S |
| s39 | 22.25581395 | S | S |
| s40 | 22.80952381 | S | S |
| s41 | 11.5 | S | S |
| s42 | 23.3902439 | S | S |
| s43 | 22.25581395 | S | S |
| s44 | 21.72727273 | S | S |
| s45 | 22.25581395 | S | S |
| s46 | 22.25581395 | S | S |
| s47 | 22.25581395 | S | S |
| s48 | 22.25581395 | S | S |
| s49 | 0.098901099 | R | R |
| s50 | 22.25581395 | S | S |
| s51 | 22.25581395 | S | S |
| s52 | 13.28571429 | S | S |
| s53 | 22.25581395 | S | S |
| s54 | 18.60784314 | S | S |
| s55 | 65.66666667 | S | S |
| s56 | 22.25581395 | S | S |
| s57 | 0.011122346 | R | R |
| s58 | 0.078748652 | R | R |
| s59 | 22.25581395 | S | S |
| s60 | 12.33333333 | S | S |
| s61 | 22.25581395 | S | S |
| s62 | 15.66666667 | S | S |
| s63 | 16.85714286 | S | S |
| s64 | 22.25581395 | S | S |
| s65 | 22.25581395 | S | S |
| s66 | 22.25581395 | S | S |
| s67 | 9.309278351 | S | S |
| s68 | 10.62790698 | S | S |
| s69 | 16.24137931 | S | S |
| s70 | 10.11111111 | S | S |
| s71 | 22.25581395 | S | S |
| s72 | 0.022494888 | R | R |
| s73 | 22.25581395 | S | S |
| s74 | 22.25581395 | S | S |
| s75 | 22.25581395 | S | S |
| s76 | 21.72727273 | S | S |
| s77 | 22.25581395 | S | S |
| s78 | 20.73913043 | S | S |
| s79 | 0.026694045 | R | R |
| s80 | 22.25581395 | S | S |
| s81 | 22.25581395 | S | S |
| s82 | 22.25581395 | S | S |
| s83 | 18.60784314 | S | S |
| s84 | 4.076142132 | S | S |
| s85 | 0.338688086 | R | R |
| s86 | 22.25581395 | S | S |
| s87 | 14.15151515 | S | S |
| s88 | 13.92537313 | S | S |
| s89 | 110.1111111 | S | S |
| s90 | 4.952380952 | S | S |
| s91 | 18.60784314 | S | S |
| s92 | 22.25581395 | S | S |
| s93 | 22.25581395 | S | S |
| s94 | 22.25581395 | S | S |
| s95 | 18.60784314 | S | S |
| s96 | 22.25581395 | S | S |
| s97 | 22.25581395 | S | S |
| s98 | 22.25581395 | S | S |
| s99 | 0.051524711 | R | R |
| s100 | 0.03950104 | R | R |
| s101 | 22.80952381 | S | S |
| s102 | 22.25581395 | S | S |
| s103 | 5.451612903 | S | S |
| s104 | 22.25581395 | S | S |
| s105 | 22.80952381 | S | S |
| s106 | 22.80952381 | S | S |
| s107 | 22.25581395 | S | S |
| s108 | 7.771929825 | S | S |
| s109 | 11.5 | S | S |
| s110 | 0 | R | R |
| s111 | 22.25581395 | S | S |
| s112 | 22.25581395 | S | S |
| s113 | 11.19512195 | S | S |
| s114 | 23.3902439 | S | S |
| s115 | 21.22222222 | S | S |
| s116 | 22.25581395 | S | S |
| s117 | 22.25581395 | S | S |
| s118 | 17.86792453 | S | S |
| s119 | 9.526315789 | S | S |
| s120 | 22.25581395 | S | S |
| s121 | 36.03703704 | S | S |
| s122 | 22.80952381 | S | S |
| s123 | 22.25581395 | S | S |
| s124 | 22.25581395 | S | S |
| s125 | 7.928571429 | S | S |
| s126 | 0.02145046 | R | R |
| s127 | 22.25581395 | S | S |
| s128 | 15.39344262 | S | S |
| s129 | 22.25581395 | S | S |
| s130 | 0.007049345 | R | R |
| s131 | 14.15151515 | S | S |
| s132 | 22.25581395 | S | S |
| s133 | 0.100110011 | R | R |
| s134 | 2.448275862 | S | R |
| s135 | 30.25 | S | S |
| s136 | 23.3902439 | S | S |
| s137 | 0.034126163 | R | R |
| s138 | 0.009081736 | R | R |
| s139 | 0.123595506 | R | R |
| s140 | 10.36363636 | S | S |
| s141 | 0.019367992 | R | R |
| s142 | 32.33333333 | S | S |
| s143 | 0.044932079 | R | R |
| s144 | 0.005025126 | R | R |
| s145 | 12.51351351 | S | S |
| s146 | 22.80952381 | S | S |
| s147 | 49 | S | S |
| s148 | 49 | S | S |
| s149 | 51.63157895 | S | S |
| s150 | 13.92537313 | S | S |
| s151 | 22.25581395 | S | S |
| s152 | 65.66666667 | S | S |
| s153 | 0.006036217 | R | R |
| s154 | 23.3902439 | S | S |
| s155 | 11.65822785 | S | S |
| s156 | 0.016260163 | R | R |
| s157 | 0.033057851 | R | R |
| s158 | 0.024590164 | R | R |
| s159 | 0.016260163 | R | R |
| s160 | 9.752688172 | S | S |

The confusion matrix generated by the test result data is shown in Table 2:

TABLE 2

predicting result

| | | |
|---|---|---|
| confusion matrix | R | S |

| | | | |
|---|---|---|---|
| real result | R | 30 | 1 |
| | S | 0 | 129 |

Assuming TP (True Positive) represents the number of true positive cases, FP (False Positive) represents the number of false positive cases, FN (False Negative) represents the number of false negative cases, and TN (True Negative) represents the number of true negative cases. Precision refers to the proportion of positive samples in the positive case determined by the classifier. The recall rate refers to the proportion of predicted positive cases to the total positive cases. Accuracy refers to the proportion of correct judgments made by the classifier on the entire sample. F1 score is the harmonic mean of accuracy and recall, with a maximum of 1 and a minimum of 0. The calculation results of each indicator are as follows:

$$precision = \frac{TP}{TP + FP} = \frac{30}{30 + 0} = 1 \\ recall = \frac{TP}{TP + FN} = \frac{30}{30 + 1} = 0.968 \\ accuracy = \frac{TP + TN}{TP + FP + TN + FN} = \frac{30 + 129}{30 + 1 + 129 + 0} = 0.994 \\ F 1 = \frac{2 \times precision \times recall}{precision + recall} = 0.984$$

The above examples only express the embodiments of the present invention, and their description is more specific and detailed, but they cannot be understood as a limitation on the scope of the invention patent. It should be pointed out that for an ordinary technical person skilled in the art, several deformations and improvements can be made without departing from the concept of the present invention, all of which fall within the scope of protection of the present invention. Therefore, the scope of protection of the present invention patent should be based on the claims.

1. A system of predicting sensitivity of Klebsiella against MeropeneM, comprising: a computing unit, said computing unit comprises a computer readable storage medium on which is stored a computer program, wherein an Exp (−k) power value calculation method is implemented when said computer program is executed by a processor, said Exp(−k) power value calculation method comprises the following computing steps:
S1: k value is calculated according to formula I:
$$k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - 0.46 \times ( \frac{C 5 - 0.196}{0.67} ) \qquad Formula I$$
S2: Exp(−k) power value with natural constant e as base and −k as exponent is calculated;
in formula I,
C1 is the number of mphA gene copies in a candidate Klebsiella strain,
C2 is the number of marA gene copies in a candidate Klebsiella strain,
C3 is the number of Klebsiella pneumoniae KpnE gene copies in a candidate Klebsiella strain,
C4 is the number of KPC-1 gene copies in a candidate Klebsiella strain,
C5 is the number of floR gene copies in a candidate Klebsiella strain.

2. The system of predicting sensitivity of Klebsiella against MeropeneM according to claim 1, wherein the system comprises a result output unit, the computing unit transmits the calculated Exp(−k) power value to the result output unit, the result output unit recognizes Exp(−k) power value and outputs result.

3. The system of predicting sensitivity of Klebsiella against MeropeneM according to claim 1, wherein said natural constant e=2.718281828459045.

4. The system of predicting sensitivity of Klebsiella against MeropeneM according to claim 2, wherein the result output unit outputs resistant result R when recognizing Exp(−k) power value <1, and the result output unit outputs sensitive result S when recognizing Exp(−k) power value ≥1.

5. The system of predicting sensitivity of Klebsiella against MeropeneM according to claim 2, wherein the result output unit and the computing unit are communicated through a data-path, Exp(−k) power value calculated by the computing unit is transmitted to the result output unit through the data-path.

6. The system of predicting sensitivity of Klebsiella against MeropeneM according to claim 4, wherein the result output unit and the computing unit are communicated through data-path, Exp(−k) power value calculated by the computing unit is transmitted to the result output unit through data-path.

7. The system of predicting sensitivity of Klebsiella against MeropeneM according to claim 4, said sensitive result S refers to that the candidate Klebsiella strain is sensitive to MeropeneM, and said resistant result R refers to that the candidate Klebsiella strain is resistant against MeropeneM.

8. The system of predicting sensitivity of Klebsiella against MeropeneM according to claim 1, wherein:
the system comprises an experiment unit and a data input unit;
the experiment unit and the data input unit are communicated through a data-path;
the experiment unit outputs experiment results which are transmitted to the data input unit and transformed to independent variables;
said data input unit and said computing unit are communicated through the data-path; and
independent variables are transmitted to the computing unit.

9. The system of predicting sensitivity of Klebsiella against MeropeneM according to claim 8, wherein said independent variables include: values of C1, C2, C3, C4, C5.

10. The system of predicting sensitivity of Klebsiella against MeropeneM according to claim 8, wherein said experiment results comprise: the number of mphA gene copies in the candidate Klebsiella strain, the number of marA gene copies in the candidate Klebsiella strain, the number of Klebsiella pneumoniae KpnE gene copies in the candidate Klebsiella strain, the number of KPC-1 gene copies in the candidate Klebsiella strain, the number of floR gene copies in the candidate Klebsiella strain.

11. A method of predicting sensitivity of Klebsiella against MeropeneM, comprising:
S1: k value is calculated according to formula I:
$$k = - 1.44 + 0.708 \times ( \frac{C 1 - 0.223}{0.757} ) - 0.66 \times ( \frac{C 2 - 0.951}{0.09} ) + 0.088 \times ( \frac{C 3 - 0.952}{0.103} ) + 3.048 \times ( \frac{C 4 - 0.469}{1.09} ) - 0.46 \times ( \frac{C 5 - 0.196}{0.67} ) \qquad Formula I$$
S2: Exp(−k) power value with natural constant e as base and −k as exponent is calculated;
in formula I,
C1 is the number of mphA gene copies in a candidate Klebsiella strain,
C2 is the number of marA gene copies in a candidate Klebsiella strain,
C3 is the number of Klebsiella pneumoniae KpnE gene copies in a candidate Klebsiella strain,
C4 is the number of KPC-1 gene copies in a candidate Klebsiella strain,
C5 is the number of floR gene copies in a candidate Klebsiella strain;
a predicting result corresponding to Exp(−k) power value<1 is the candidate Klebsiella strain sensitive to MeropeneM, and a predicting result corresponding to Exp(−k) power value≥1 is the candidate Klebsiella strain resistant against MeropeneM.

12. The method of predicting sensitivity of Klebsiella against MeropeneM according to claim 11, wherein said natural constant e=2.718281828459045.

13. The method of predicting sensitivity of Klebsiella against MeropeneM according to claim 11, wherein the number of mphA, marA, Klebsiella pneumoniae KpnE, KPC-1, floR gene copies in the candidate Klebsiella strain are obtained through a second-generation high-throughput sequencing method.

14. The method of predicting sensitivity of Klebsiella against MeropeneM according to claim 13, wherein the number of gene copies in the candidate Klebsiella strain=depth of gene contigs/depth of genome contigs.

15. The method of predicting sensitivity of Klebsiella against MeropeneM according to claim 14, wherein:
said genome contigs is a longest contigs segment assembled from sequencing results by SPAdes v3.13.0 software;
said depth of genome contigs is a depth of genome contigs calculated by SPAdes v3.13.0 software; and
said depth of gene contigs refers to a sum of depths of each contig which has said gene copies and said gene is located on.

16. The method of predicting sensitivity of Klebsiella against MeropeneM according to claim 15, wherein each contig which has said gene copies is annotated by blat (v. 36) software and diamond (v2.0.4.142) software through CARD database alignment between said gene cds and protein sequence.

17. The method of predicting sensitivity of Klebsiella against MeropeneM according to claim 15, wherein depths of each contig which has said gene copies and said gene is located on are calculated through SPAdes v3.13.0 software.
